(19) United States
(12) Patent Application Publication
(10) Pub. No.: US 2006/0040911 A1
(43) Pub. Date: Feb. 23, 2006

(54) Method for preventing and/or treating the cardiovascular and hepatic diseases induced by hyperlipidemia which comprises administered an effective amount of bioflavonoids extract derived from fructus crataegus (lipid metabolism and fructus crataegus)

(76) Inventors: Benedict Schue Liao, Walnut, CA (US); Judy Fu-Chuan Liao, Monterey Park, CA (US); Alex Liao, Walnut, CA (US); Austin Liao, Walnut, CA (US); Burton Arthur Liao, Walnut, CA (US); Su-Hsin Liao-Tung, Iowa City, CA (US); Susan Liao-Nieng, Chicago, IL (US); Cathy Nieng, Chicago, IL (US); Su-Lien Liao-Chen, Baltimore, MD (US); Schue-Yuan Liao, Baltimore, MD (US)

Correspondence Address: DR. BENEDICT S. LIAO, M.D., SUITE 206, 600 N. GARFIELD AVE., MONTEREY PARK, CA, 91754, US

(21) Appl. No.: 11/226,862

(22) Filed: Sep. 13, 2005

Related U.S. Application Data
(63) Continuation of application No. 10/337,434, filed on Jan. 6, 2003.

(51) Int. Cl. A61K 31/33

(52) U.S. Cl. 514/183

(57) ABSTRACT

A method for treating and/or preventing the cardiovascular and hepatic diseases induced by hyperlipidemia which comprises administered thereto an effective amount of bioflavonoids extract derived from fructus crataegus such as; rutin, quercetin, kaempferol and vitexin or a mixture thereof.

### CROSS REFERENCES TO RELATED APPLICATIONS

### REFERENCES CITED

U.S. Patent Documents

| Patent No. | Date | Inventors | Class |
|---|---|---|---|
| 6180660 | January, 2001 | Whitney et al. | 514/451 |
| 6238673 | May 2001 | Howard et al. | 424/195.1 |
| 20010046963 | November 2001 | Wenzel et al. | 514/27 |
| 6225338 | May 2001 | Romanczyk et al. | 514/453 |
| 6221357 | April 2001 | Bok et al. | 514/27 |
| 4346227 | August 1982 | Terehara et al. | 560/119 |
| 5354772 | October 1994 | Kathawala et al. | 514/414 |

### OTHER REFERENCES

Mark H. Beers and Robert Berkow, Editors, Hyperlipidemia; Clinical, Biochemical and Pharmacological aspects; The Merck manual of Diagnosis and Therapy, 17^{th} edition, Merck Research Laboratories publishing: pp. 200-212 (1999).
Ross, R. et al., The Pathogenesis of atherosclerosis: a perspective for the 1990s, Nature; 362, pp. 801-809(1993).
Stary, H. C. and Chandler A. B. et al., A definition of advanced typed of atherosclerotic lesions and histological classification of atherosclerosis. A report from the Committee on Vascular
lesions of the Council on Atherosclerosis, American Heart Association. Circulation, 92: 1355-1374 (1995).
Lubert, Stryer. Author, Textbook of Biochemistry, 3^{rd} edition, Chapter 23, Biosynthesis of lipids; W. H. Freeman and Co. Publishing: pp. 554-566 (1988).
Witiak, D. T. and Feller, D. R. et al., Antilipidemic Drugs: Medicinal, Chemical and Biochemical Aspects; Elsevier, pp. 159-195 (1991).
Gerald K. McEvoy, Editor, Antilipidemic agents: Clinical, Biochemical and Pharmacological aspects, Drug Information: American Society of Health System Pharmacists publishing: pp. 1430-1468 (1998) and pp. 1705-1750 (2001)
Revilla E, and Ryan J M. Et al.; Analysis of several phenolic compounds with potential antioxidant properties in grape extracts and wines by high-performance liquid chromatography-Photodiode array detection without sample preparation,: Journal of Chromatographia, 6,881(1-2); pp.461-469 (2000)
E. Reinhard, Tubingen Editor-in-Chief, Ammon, H. P. T. and Haendel, M. et al., Planta Medica, Toxicology and Pharmacology of Crataegus: Journal of Medical Plant Research: 43 (2) pp. 105-120,: 43(3) pp. 210-239: 43 (4) pp. 316-323 (1981)
D. A. Rakotoarison et al. Antioxidant activities of Polyphenolics extracts from flowers of Craetaegus Monogyna. Pharmazie; 52: pp.60-64 (1997)
Bayler T., et al., Bioflavonoids and its anti-inflammatory effects; Phytochemistry, 28, pp. 2373-2378 (1989)
Kaul, T. N. and Elliot Middleton et al., Antiviral effect of Citrus Flavonoids on human viruses, Journal of Medical Virology: 15; pp 71-79 (1985)
Saija, A. and Scalese, M. et al., Flavonoids, Quercetin, Hesperetin, Naringenin, and Rutin, as Anti-oxidant agents: Free Radical Biology and Medicine: vol. 19, no. 4, pp. 481-486 (1995).
Felicia V. So, and Najla Guthrie et al., Inhibition of human breast cancer cell perliferation and delay of mammary tumor-genesis by Flavonoids and citrus juices: Nutrition and Cancer, 26 (2), pp. 167-181 (1996).
Bok, S. H. and Jeong, T. S. et al., Flavonoids derived from citrus peel as collagen induced platelet aggregation inhibitor: U.S. laid open Pat. No. 6,221,357.
Nair, M. G. and Wang, H. B. et al., Method of inhibiting cyclooxygenase and inflammation using cyanidins: U.S. laid open Pat. No. 10,002,407.
Shanthi S. et al., Hypolipidemic activity of tincture of Crataegus in rats: Indian Journal of Biochemistry and Biophysics; 31(2), pp. 143-146 (1994).
Rajendran S. and Deepalakshmi P. D. et al., Effect of tincture of Crataegus on the LDL-Receptor Activity of hepatic plasma membrane of rats fed an atherogenic diet. Atherosclerosis: 123: pp. 235-241(1996)
Wang S. L. and Li, Y. D. et al., Inhibition of 3-HMG CoA Reductase activity in hepatic and intestinal mucosal cells in guinea pig fed with concentrated aqueous extract of Crataegus Pinnatifida; Journal of Traditional Chinese Medicine: 7;(8): pp. 483-484 (1987)
Zhao J, Wang J, Chen Y, and Agarwa R.: Antitumor promoting activity of a polyphenolic fraction isolated from grape seeds in the mouse skin two-stage initiation-promotion protocol and identification of procyanidin B5-3 -gallate as the most effective antioxidant constituent. Carcinogenesis, September 1999; 20(9); pp. 1737-1745.
Guan Y, Zhao S. et al., Blood lipid tablets in the treatment of Hyperlipidemia: Journal of Traditional Chinese Medicine: 15 (3), pp 178-179 (1995).
Scambia, G. and Ranelletti, F. O. et al., Quercetin inhibits the growth of a Multidrug-Resistant Estrogen Receptor—Negative MCF-7 Human Breast Cancer cell line Expressing Type II Estrogen-Binding sites; Cancer Chemotherapy, Pharmacology: 28, pp. 255-258 (1991)
Scambia, G. and Ranelletti, F. O. et al., Type II Estrogen binding sites in a Lymphoblastoid cell line and Growth-inhibitory effect of Estrogen, Anti-Estrogen and Bioflavonoids; International Journal of Cancer: 46: pp. 1112-1116 (1990).
Singhal, R. L. and Yeh, Y. A. et al., Quercetin Down-Regulates Signal Transduction in Human Breast Cancer cells: Biochemistry, Biophysicis Research Commun.: 208, pp 425-431, (1995)
Castillo, M. H. and Perkins, E. et al., The effect of the Bioflavonoid—Quercetin on Squamous cell Carcinoma of Head and Neck origin, American Journal of Surgery: 158, pp. 351-355 (1989).
Ranelletti, F. O. and Ricci, R. et al., Growth Inhibitory effect of Quercetin and presence of Type-II Estrogen-binding sites in human colon cancer cell lines and primary colorectal tumors; International Journal of Cancer: 50, pp. 486-492 (1992).
Verma, A K. Johnson, J A. et al., Inhibition of 7,12-Dimethylbenza-anthracene and N-Nitrosomethylurea-induced rat Mammary Cancer by Dietary Flavonoid Quercetin; Cancer Research: 48, pp. 5754-5758 (1988).
Ammon, H. P. T., and Haendel, M. et al., Crataegus Toxicology and Pharmacology, Planta Medica: 43(2): pp. 105-120, 43(3): pp. 210-239, 43(4): pp. 316-323 (1981).
Schussler, M. and Holzl, J. et al., Increasing Cardiac Perfusion with Quercetin, Rutin, and Vitexin in guinea pig heart: Arzneimittelforschung, 45(8): pp. 842-845 (1995).
Manach, C. et al., Bioavailability, Metabolism, and Physiological impact of 4-oxo-Flavonoids: Nutrition Research 16: pp. 517-544 (1996).
Hertog, M. G. L. et al., Dietary Anti-oxidant Flavonoids and risk of coronary heart disease: Lancet: 342: pp. 1007-1011 (1993).
Peterson, G, and Barnes, S.: Genistein Inhibition of the growth of Human Breast cancer cells: Independence from Estrogen receptors and the Multi-drug resistance gene: Biochemistry, Biophysics Research Commun.: 179, pp. 661-667 (1991)
Barnes, S. et al., Effect of Genistein on In Vitro and In Vivo Models of Cancer: Journal of Nutrition: 125, pp. 777S-783S (1995).
Guthrie, N. and Moffatt, M. et al., Inhibition of proliferation of Human Breast Cancer cells By Naringenin, a Flavonoid in Grapefruit: National Forum Breast Cancer, Montreal. P 119 (1993).
Kandaswami, C. and Perkins, E. et al., Antiproliferative effects of Citrus Flavonoids on Human Squamous Cell Carcinoma in Vitro: Cancer Lett.: 56, pp. 147-152 (1991).
Hulcher, F. H. and Oleson W. H. et al., Simplified Spectrophotometric assay for microsomal 3-hydroxy-3-methyl-glutaryl-Co enzymeA reductase by measurement of coenzyme A. Journal of Lipid Research; 14, pp. 625-641(1973).
Fogt F. and Nanji A. et al., Alterations in nuclear ploidy and cell phase distribution of rat liver cells in experimental alcoholic liver disease: relationship to antioxidant enzyme gene expression. Toxicology and applied Pharmacology; 136 (1) pp. 87-93(1996).
Keegan A. and Martini R. et al,. Ethanol-related liver injury in the rat: a model of steatosis, inflammation and pericentral fibrosis, Journal of Hepatology; 23 (5), pp 591-600 (1995).
Fleming, Thomas. Chief editor, Crataegus laevigata; Physician Desk Reference for Herbal Medicine, 2 nd edition, Medical Economics Co. publishing; pp. 271-275(2000).

### SUMMARY OF THE INVENTION

### FIELD OF THE INVENTION

The present invention relates to a method for preventing and/or treating the cardiovascular and hepatic diseases induced by elevated plasma lipid level. These include hyperlipidemia, hypercholesterolemia, atherosclerosis, arteriosclerosis, angina pectoris, stroke (cerebro-vascular accident) and liver diseases in a mammal, the method comprises administered an effective amount of bioflavonoids extract derived from hawthorn berry (fructus crataegus) such as rutin, quercetin, kaempferol, vitexin or a mixture thereof.

### BACKGROUND OF THE INVENTION

According to the recent studies and reports, coronary vascular diseases such as (e.g.) hyperlipidema, hypercholesterolemia, atherosclerosis, stroke (cerebro-vascular accident) have been the number one cause of deaths in the America. The study of Ross R., et al, [Nature, 362, 801-809 (1993)] confirmed that the elevated serum lipids e.g., cholesterols and triglycerides can cause deposition of fat and macrophage foam cells onto vascular endothelium and arterial walls and progressively develop into atheroma and atherosclerosis. The histo-pathological classification of the atherosclerotic lesions by Stary H. C., et al. (Circulation, 92: 1355-1374(1995)) showed that there are six types of lesions. The initial type I lesion contains only macrophages and macrophage foam cells in the vascular wall; the type II lesion contains macrophage foam cells and lipid-laden cells (fatty streak) in the smooth muscle cells. The type III lesion is atheroma, which contains lipid-laden cells (fatty streak) and scattered collections of extracellular lipid droplets and particles in the smooth muscle cells of the arterial wall. Type IV lesion contains a more disruptive core of extracellular lipid; the type V lesion contains largely calcified and some fibrous connective tissue and little or no accumulation of lipid and calcium. The type VI lesion contains fissure, hematoma and thrombus in the vascular wall.

The two major plasma lipids, including cholesterol (or total cholesterol [TC]) and triglycerides, are bound to proteins and transported as macromolecular complexes called lipoproteins. The major lipoprotein classes including Chylomicrons, very low density lipoproteins (VLDL), low density lipoproteins (LDL), and high density lipoproteins (HDL), the chylomicrons are the largest lipoproteins, carry exogenous triglycerides from the intestine via thoracic duct to the venous stream. In the capillaries of adipose and muscle tissue, 90% of the chylomicron triglyceride is removed by a specific group of lipases. Fatty acid and glycerol, derived from hydrolysis of chylomicrons, enter the adipocytes and muscle cells for energy use or storage. The liver then removes the remnant chylomicron particles, VLDL carries endogenous triglyceride primarily from the liver to the peripheral sites (adipocytes and muscle cells) for storage or use. The same lipases that act on chylomicrons quickly degrade endogenous triglyceride in VLDL, giving rise to intermediate density lipoproteins (IDL) that are shorn of much of their triglyceride and surface apoproteins. This IDL is degraded further by removal of more triglyceride, giving rise to LDL, which is the main source plasma LDL. The liver removes 70% of LDL from blood stream and active receptor sites have been found on the surfaces of hepatocytes and other cells that specifically bind to apolipoprotein B (apo B, the ligand associated with LDL that binds with LDL receptors)and remove most LDL from circulation. A small but important amount of LDL appears to be removed from circulation by non-LDL receptor pathways, including uptake by scavenger receptors on macrophages that may migrate into arterial walls, where they may become the foam cells of atherosclerotic plaques. Hypercholesterolemia can result either from overproduction or defective clearance of VLDL or from increased conversion of VLDL to LDL. (Mark H. Beers and Robert Berkow, Eds, Hyperlipidemia; Clinical, Biochemical and Pharmacological aspects; The Merck manual of Diagnosis and Therapy, 17th edition, Merck Research Laboratories publishing, pp. 200-212 (1999))

It is conceivable that decreasing the plasma cholesterol and lipid level would decrease the chance of atherosclerosis and arteriosclerosis. The prevention of hyperlipedmia and/or hypercholesterolemia can be resulted from either reducing the amount of the alimentary ingestion of cholesterol and lipids. Or inhibiting the absorption of cholesterol by inhibiting the activities of the convertal and/or acyl CoA-transferase enzymes (ACAT), thirdly, facilitating the rate of the degradation and clearance of cholesterol and lipids in the blood stream.

It is well known that inhibiting the activity of the 3-hydroxy-3-methyl-glutaryl CoA (3-HMG CoA) reductase will reduce the rate of mevalonic acid, (an intermediate) and cholesterol biosynthesis. (Textbook of Biochemistry, Lubert Stryer, 3rd edition, Freeman Publishing, pp. 554-564, 1988) Substances and Chemicals which have effectively inhibited the activity of the 3-HMG CoA reductase will be effective in preventing and/or treating the hypercholesterolemia and hyperlipidemia in mammals.

Furthermore, low density lipoprotein (LDL) in the serum contains a large amount of the cholesterol esters. Cholesterol esterification is promoted by acyl-CoA-cholesterol-0-acyltransferase (ACAT) which leads to foam cell formation in the vascular wall and eventually develops of atherosclerosis. On the contrary, the ACAT enzyme inhibitor will not only reduce the concentration of the LDL in the serum but also prevent atherosclerosis and arteriosclerosis in mammals. (Witiak, D. T. and Feller, D. R.; Antilipidemic Drugs: Medicinal, Chemical and Biochemical aspects, Elsevier, pp 159-195(1991)) (Gerald K. McEvoy Ed. Antilipidemic agents: Chemical, Biochemical and Pharmacological aspects, Drug Information: American Society of Health System Pharmacists publishing, pp. 1430-1468 (1998), and pp. 1705-1750 (2001))

Several medicines acting as the inhibitor of 3-HMG-CoA reductase have been developed e.g.; Lovastatin and Simvastatin (Merck Co.,U.S.A.) and Pravastatin (Sankyo, Japan), but due to the serious side effects on long term administration, the continual usage of Vastatins have major drawbacks. The central nervous system side effects include perivascular hemorrhage, edema, mononuclear cell infiltration, fibrin deposit, necrosis of small vessels and optic nerve degeneration. Vastatins not only enhance the activities of LDL receptor but also increase creatine kinase in the liver that in term could develop into acute renal failure secondary to rhabdomyolysis. (Gerald K. McEvoy, Ed. Anti-lipidemic Agents: Chemical, Biochemical and Pharmacological Aspects, Drug Information, American Society of Health System Pharmacists publishing, pp 1705-1750 (2001)) Thereby, the need to develop a nontoxic and inexpensive inhibitor of 3-HMG-CoA reductase is highly appreciated.

Overproduction of the VLDL by the liver may be caused by obesity, diabetes mellitus, excess alcohol consumption, nephrotic syndrome, or genetic disorders; each condition can result in increased LDL and TC level and often is associated with hyper-triglyceridemia. Fatty liver is also a type of hyperlipidemia induced disease, in which a large amount of lipids is deposited into hepatocytes of liver tissue and can result in increasing LDL and TC level and elevation of Serum Glutamate-oxaloacetate Transaminase (SGOT), Serum Glutamate-Pyruvate Transaminase (SGPT) and Gamma-Glutamyl-Transpeptidase (gamma-GTP) or (GGTP).

Bioflavonoids are group of naturally occurring compounds, which have a common flavone nucleus composed of two benzene rings linked through a heterocyclic-pyrone ring. They are found plentifully in various plants, vegetables, fruits (such as; citrus fruits, grapes), food products (such as buckwheat and oatmeal) and dyes of natural origin. Bioflavonoids exhibit various biochemical and pharmacological activities including anti-oxidative, anti-inflammatory, anti-cancer, anti-viral, and anti-platelet aggregation. (D A. Rakotoarison et al. Antioxidant activities of polyphenolic extracts from flowers of Crataegus monogyna. Pharmazie; 52: pp. 60-64 (1997)), (Bayler, T.,et al., Phytochemistry 28, 2373-2378 (1989)), (Goda, Y., et al., Chem. Pharm. Bull. 40, pp. 2455-2457(1992)), (T N. Kaul and Elliott Middleton et al., Antiviral effect of Citrus flavonoids on human viruses, Journal of medical virology: 15; pp 71-79(1985)), (A. Saija and M. Scalese et al., four flavonoids, Quercetin, hesperetin, naringenin, and rutin, as anti-oxidant agents; Free radical biology and medicine, vol. 19, no. 4, pp 481-486(1995)), (Felicia V. So. and Najla Guthrie et al., Inhibition of human breast cancer cell proliferation and delay of mammary tumor-genesis by flavonoids and citrus juices, Nutrition and cancer vol. 26,no. 2, pp. 167-181(1996)), (S H. Bok and T S. Jeong et al., Flavonoides derived from citrus peel as collagen induced platelet aggregation inhibitor, U.S. Laid open Pat. No.: 6,221,357), (M G. Nair and H B. Wang et al., method of inhibiting cyclooxygenase and inflammation using cyanidin, U.S. Laid-open Pat. No.: 10,002,407)

In the recent studies and reports by Shanthi S. et al (Indian Journal of Biochem. Biophy. 31: 2; pp. 143-146 (1994)), and Rajendran S. and P D. Deepalakshmi et al., (Atherosclerosis; 123: pp. 235-24 (1996)) both indicated that there are hypolipidemic activity of tincture of Crataegus Oxyacantha and increased the activity of LDL-receptor in hepatic cell membrane in rats. The reductions of cholesterol and triglycerides were progressive in LDL and VLDL lipoprotein fractions.

Further study from Wang, S L. and Li, Y D. et al., revealed that 3-HMG CoA reductase activity has been reduced in hepatic and intestinal mucosal cells by 70% and 67% respectively, in guinea pig fed with concentrated aqueous extract of Crataegus Pinnatifida. (Journal of the Traditional Chinese Medicine: 7 (8); pp. 483-484 (1987)). Also Guan, Y. and Zhao, S. et al., conducted in a clinical trial of 130 hyperlipidemic subjects and achieved impressive results with a combination of Chinese herbs and Crataegus Pinnatifidae. Reduction in serum total cholesterol and triglycerides were observed in 87% and 80% of subjects respectively. (Journal of Traditional Chinese Medicine; 15(3); 178-179 (1995)), (Chen, J. D. and Wu, Y. Z. et al., World Review Nutrition and Diet, 77: 147-154(1995))

Quercetin (3,5,7,3′,4′ penta-hydroxy flavone) also has anti-cancer activities against breast cancer, colon cancer cells, lymphoblastoid cell lines and squamous cancer cell lines and anti-viral activities against herpes simplex type I, polio virus type I, para-influenza type-3, and respiratory syncytial virus. (Scambia, G. and Ranelleti, F. O. et al., Cancer Chemotherapy, Pharmacology: 28, pp. 255-258 (1991)), (Singhal, R. L. and Yeh, Y. A. et al., Biochem. Biophys. Research Commun. 208, pp. 425-458 (1995)), (Ranelleti, F. O. and Ricci, R. et al., International J. of Cancer: 50; pp. 486-492 (1992)), (Scambia, G. and Ranelleti, F. O. et al., International J. of Cancer: 46; 1112-1116 (1990)), (Castillo, M H. and Perkins, E. et al., American J. Surgery 158: pp. 351-355 (1989)), (Verma, A. K. and Johnson, J. A. et al., Cancer Research 48: pp. 5754-5758 (1988)), (Kaul, T. N. and Middleton, E. J. et al., Journal of Medical Virology 15: pp. 71-79 (1985)), (Japanese Laid-open Patent No. 4-234320)

Rutin, a glycosylated quercetin, (Quercitin-3-rutinoside) is decomposed in the intestine by microorganisms and absorbed in the intestine, Vitexin (Apigenin, Orientoside, or Flavone, 8-D-glucosyl-4′,5,7-tri-hydroxy-), and Kaempherol (3,5,7,4′ tetra-hydroxy-flavone) also have anti-hypertensive properties and increase coronary and cardiac perfusion. (Ammon, H. P. T., and Haendel, M. et al., Crataegus toxicology and Pharmacology, Planta medica: 43(2): pp. 105-120, 43(3): pp. 316-322, and 43(4): pp. 210-239 (1981)), (Schussler, M. and Holzl, J. increasing cardiac perfusion with quercetin, rutin and vitexin in guinea pig heart, Arzneimittelforschung, 45(8): pp. 842-845 (1995)), (Manach, C. et al., bioavailability, metabolism, and physiological impact of 4-oxo-flavonoids. Nutrition research 16: pp 517-544(1996)). Furthermore, Hertog et al. reported that high intake of rutin and quercetin in the food product may reduce coronary heart disease related death rate of elderly patients (M. G. L. Hertog et al., dietary antioxidant flavonoids and risk of coronary heart disease, Lancet: 342: pp. 1007-1011 (1993)).

The present inventors have discovered that bioflavonoids derived from hawthorn berry, such as rutin, quercetin, kaempherol, and vitexin are effective in treating and/or preventing elevated plasma lipid related diseases. They have remarkably reduced plasma cholesterol and triglycerides; inhibit the activities of 3-HMG-CoA reductase and ACAT; prevent the formation and accumulation of lipid containing macrophage onto arterial endothelial walls and prevent lipid deposition in the hepatic cells which may lead to hepatic dysfunction in mammals.

### SUMMARY OF THE INVENTION

The primary objective in the present invention is to provide a treatment for elevated plasma lipid related cardiovascular diseases such as hyperlipidemia, hypercholesterolemia, arteriosclerosis, atherosclerosis, angina pectoris, stroke, and hepatic disease in mammals

Therefore, the present invention relates to a method for inhibiting and/or treating and/or preventing cardiovascular and hepatic diseases induced by hyperlipidemia in a mammal, which comprises administered thereto an effective amount of bioflavonoid extract derived from Fructus Crataegus (hawthorn berry), such as rutin, quercetin, kaempherol, vitexin, or a mixture thereof.

### BRIEF DESCRIPTION OF THE DRAWINGS

The objects of the present invention will become increasingly apparent by reference to the following description and the drawings.

FIGS. IA, IB, IC, ID, IE and IF show the thoracic arterial endothelial wall of the rabbits administered with Kaytee rabbit diet with 1% cholesterol; 1% cholesterol plus 1.5 mg/kg Simvastatin; 1% cholesterol plus 0.15% rutin; 1% cholesterol plus 0.15 % Quercetin; 1% cholesterol plus 0.15% Kaempherol; and 1% cholesterol plus 0.15% vitexin respectively; and

FIGS. 2A, 2B, 2C, 2D, 2E and 2F represent the microscopic features of the liver of the rabbits administered with Kaytee rabbit diet with 1% cholesterol; 1% cholesterol plus 1.5 mg/kg Simvastatin.; 1% cholesterol plus 0.15% rutin; 1% cholesterol plus 0.15% Quercetin; 1% cholesterol plus 0.15% Kaempherol; and 1% cholesterol plus 0.15% vitexin, respectively.

### DETAILED DESCRIPTION OF THE PREFERRED EMBODIMENTS

### DETAILED DESCRIPTION OF THE INVENTION

As mention above plasma lipids include cholesterol and triglycerides in the blood stream of a mammal, and the term “elevated plasma lipids” means that plasma lipids are higher than normal level. Also the term “elevated plasma lipid level related diseases” are caused by elevated or high plasma lipid, such as hyperlipidemia, hypercholesterolemia, atherosclerosis, arteriosclerosis, angina pectoris, stroke (cerebro-vascular accident) and fatty liver.

Rutin (C.sub.27.H.sub.30. O.sub.16, Molecular weight of 610.52, glycosylated quercetin, or quercetin-3-rutinoside); Quercetin (C.sub.15. H.sub.10. O.sub.7, Molecular weight of 302.24, or 3,5,7,3′,4′ penta-hydroxy flavone); Kaempherol (C.sub.15. H.sub.10. O.sub.6, Molecular weight of 286.24, or quercetin-3-rhamnoside); Vitexin (Apigenin)(C.sub.21.H.sub.20.O.sub.10, Molecular weight of 432.38, or 8-D-glucosyl-4′,5,7 trihydroxy-flavone) (Merck Index 13rd Edition 2001) may be extracted from various plants, vegetables and fruits, such as citrus fruits, hawthorn berry, and also can be synthesized in accordance with the conventional process described by Seka, Prosche and Monatsh., 69,284 (1936) and Zemplen, Bognar in Ber., 1043 (1943). and EINECS 222-963-8, Journal of European Communities; June 1990.

For example, rutin may be found in hawthorn berries, flowers, leaves, stems, and roots in an amount ranging from 0.2 to 5 wt. % (PDR Herbal Medicines, 2nd Edition 2000). Rutin, quercetin, kaempherol and vitexin may be extracted from hawthorn berry by using a suitable solvent, such as water or aqueous ethanol alcohol under high temperature and pressure. The other method is using aqueous solution of ½ N Ca (OH) sub.2 or NaOH, and then the crude extract and precipitates may be collected after neutralization. Furthermore, the dry powders of hawthorn berries, leaves, stems, flowers and roots may also be used. Generally, content of rutin, quercetin, kaempherol and vitexin in the berries is 5%, 3%, 2%, and 0.5% respectively.

Rutin, quercetin, kaempherol and vitexin not only possess an inhibitory, but also exert a therapeutic effect on elevated plasma lipid level related diseases, such as hyperlipidemia, hypercholesterolemia, atherosclerosis, arteriosclerosis, stroke (cerebro-vascular accident), angina pectoris and hepatic disease, such as fatty liver and fatty degeneration. Furthermore, rutin, quercetin, kaempherol, and vitexin exhibit no toxicity and no adverse effects on hematopeutic, renal, hepatic systems when they are administered orally to a mouse at a dose of 1500 mg/kg, 125 mg/kg, 1000 mg/kg, 500 mg/kg, respectively, which corresponds to an oral administered dose of 50 to 150 gm. of hawthorn berry extract for a person weighing 50 kg.

The present invention also provides a pharmaceutical composition for inhibiting the formation of fatty streak onto the arterial endothelial wall, which comprise hawthorn berry extract as an active ingredient plus pharmaceutically acceptable excipients, carriers or diluents.

The hawthorn berry extract of the present invention may be prepared in accordance with any conventional method by using suitable solvents, such as water or lower alcohol (ethanol) and an aqueous alkali or alkaline earth-metal hydroxide solution such as Ca (OH) sub.2 or NaOH solution. For example, 0.5 to 1 N of 1-10 liters of a solvent is added to 1 kg of dried hawthorn berry and the mixture is kept at a temperature ranging from 25 to 70 degrees C. for a period ranging from 1 to 10 hours. The resulting extract is filtrated and concentrated to a formation of a concentrated hawthorn berry extract. For instance, if an aqueous alkali or alkaline earth metal hydroxide solution is used, the filtrate is adjusted to a PH ranging from 4.0 to 7.0 by adding an acid thereto. The resulting solution is kept at a temperature ranging from 5 to 20 degrees C. for a period ranging from 5 to 30 hours. The precipitate is then dried to obtain a hawthorn berry extract. On the other hand, when ethanol is used as a solvent, 1 to 10 liters of 30% to 100% of solvent are added to 1 kg of the dried hawthorn berry, and the mixture is kept at a temperature ranging from 25 to 70 degrees C. for a period ranging from 1 to 10 hours, then the resulting mixture is filtrated and concentrated to obtain a hawthorn berry extract.

The hawthorn berry powder may be used in the present invention in place of the hawthorn berry extract. The hawthorn berry powder may be prepared by lyophilizing or drying the solid materials from hawthorn berry according to a conventional method and powdering it to a particle size ranging from 50 to 250 mu.m.

A pharmaceutical formulation may be prepared in accordance with any of the conventional methods and procedures. In preparing the formulation, the active ingredient is preferably admixed or diluted with a carrier, or enclosed within a carrier, which may be in a form of a capsule, sachet, or other container. When the carrier serves as a diluent, it may be a solid, semisolid, or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

The pharmaceutical examples of suitable carriers, excipients and diluents are lactose, dextrose, sucrose, sorbitol, mannitol, starches, gum acacia, alginates, gelatin, calcium phosphate, calcium silicate, cellulose, methyl cellulose, micro-crystalline cellulose, polyvinylpyrrolidone, water, methyl-hydroxy-benzoates, propyl-hydroxy-benzoates, talcum, magnesium stearate and mineral oil. The formulation may additionally include fillers, anti-agglutinating agents, flavoring agents, lubricating agents, wetting agents, emulsifiers, preservatives and the like. The pharmaceutical compositions of the invention may be formulated to provide quick, sustained or delayed release of the active ingredient after its administration to a mammal by employing any of the procedures and/or methods well known in the art.

The pharmaceutical composition of the present invention contains the active ingredient in an amount ranging from 0.01 to 100 wt %, but preferably from 0.1 to 50 wt %. It can be administered via various routes such as oral, transdermal, subcutaneous, intramuscular, intravenous, inhalational, intraperitoneal and transmucosal introduction. A typical daily dose of bioflavonoids in human may range from 0.1 to 500 mg/kg of body weight, but preferably from 1.0 to 100 mg/kg of body weight and may be given in a single dose or in divided doses. The actual and exact amount of the active ingredient to be administered may vary according to patient's age, sex, body weight, disease, severity of illness and route of administration.

Furthermore, quercetin, rutin, kaempherol and vitexin may be incorporated into foods and/or beverages for the purpose of preventing and/or treating elevated plasma lipid related diseases (e.g., hyperlipidemia, hypercholesterolemia, atherosclerosis, arteriosclerosis, cerebro-vascular accident, angina pectoris and hepatic disease). The foods and beverages may include food products, meats, vegetable juices, fruit juices, snacks, confectionery (chocolates and pizza), gum, dairy products, soups, broth, pastes, sauces (such as ketchup), teas, alcohol beverages, carbonated beverages, vitamin complexes and various health foods.

The content of rutin, quercetin, kaempherol and vitexin, or a mixture thereof in a food or beverage may range from 0.1 to 10 wt %. It is therefore comprised of 1 to 100 gm of rutin, quercetin, kaempherol, vitexin or mixture thereof per 1000 ml of beverage.

### EXAMPLE 1

Preparation and Analysis of Hawthorn Berry Extract.

The hawthorn berries (Ogden, Utah. USA) were dried at room temperature and powdered to a particle size ranging from 100 to 200 mu.m.and then 50 ml of 80% ethanol was added to 10 gm of hawthorn berry powder and extracted in a water bath at 60 degree C. for 6 hours. The extract obtained was filtrated and cooled, then ethanol was added to the filtrate to a volume of 50 ml.

The above extract in a amount of 3.0 mu.l. was subjected to high performance liquid chromatography (HPLC) using prostar UV-Vis spectrophotometer lichrosorb RP-8 column (5 mu.m, 4 times, 250 mm) was pre-calibrated with 0.1 M borate sodium duodisulfate (SDS) solution and maintained at a temperature of 30 degree C. The extract was eluted with 0.1 M of borate SDS at flow rate of 0.5 ml/min. Standards solution were prepared by dissolving rutin, quercetin, kaempherol (Aldrich-Sigma Chemical Co. St. Louis, Mo. USA) and vitexin (Indofine Chemical Co. Somerville, N.J. USA) in 0.1 M borate SDS to a final concentration of 0.1, 0.2, 0.3, 0.4, and 0.5 mg/ml respectively, and subjected to HPLC under the same condition as that of above. The eluates were detected at 254 nm.(rutin), 257 nm.(quercetin), 266 nm.(Kaempherol), 270 nm.(vitexin) with UV-Vis spectrophotometer and the contents of rutin, quercetin, kaempherol and vitexin were calculated by comparing by the areas of HPLC profiles of the hawthorn berry extract and standard solution. The contents (%) of rutin, quercetin, kaempherol and vitexin in hawthorn berry extracts are depicted in Table 1.

1 TABLE I; Hawthorn berry extract contents Rutin 5.2%, Quercetin 3.1%, Kaempherol 2.3% and Vitexin 0.5%.

### EXAMPLE 2

Preparation of Hawthorn Berry Extract

(A) Method of Using Ethanol

The hawthorn berries were dried at room temperature, 300 ml of 80% ethanol was added to 100 gm of dried berry, The berry were extracted at 60 degree C. for 6 hours; the resulting extract was filtrated through cheese cloth and the filtrate was concentrated under vacuum to obtain 57 gm of syrupy extracts. The contents of rutin, quercetin, kaempherol and vitexin were examined in accordance with the method of example 1. which contained rutin 2.90 gm, quercetin 1.82 gm, kaempherol 1.31 gm, vitexin 0.285 gm.

The composition of hawthorn berry extract was confirmed by HPLC and the result is depicted in Table II

2 TABLE II Ingredient content (%), moisture 67%, fructose 4%, glucose 3%, sucrose 2.9%, rutin 5.1%, quercetin 3.2%, kaempherol 2.3%, vitexin 0.5%, others 12.1%.

(B) Method of Using NaOH

The dried hawthorn berries (Ogden, Utah. USA) in an amount of 100 gm was added to ½ N 500 ml of NaOH solution, and kept at room temperature for 3 hours while stirring. The resulting extract was obtained by filtrating through a cheese cloth, then 1 N HCl solution was added to the filtrate to adjust its PH to 4.5. The same procedure as that of above was repeated to obtain a filtrate to adjust its PH to 6.5. The resulting filtrates were kept at 6 degree C. for 12 hours and then the precipitates were collected and dried to obtain 8.8 gm and 9.8 gm powders, respectively. The compositions were confirmed by HPLC analysis which showed that hawthorn berry extracts contained rutin, (4.08 gm, 4.55 gm), quercetin (2.56 gm, 2.85 gm), kaempherol (1.84 gm, 2.05 gm), vitexin (0.40 gm, 0.45 gm) and the purity was 29.9% and 20%, respectively.

(C) Method of Using Ca (OH) sub.2

The dried hawthorn berry (Ogden, Utah. USA) in an amount of 100 gm was added to ½ N 500 ml of Ca(OH) sub.2 solution, and kept at room temperature for 3 hours while stirring.The resulting extract obtained by filtrating through a cheese cloth, and then 1 N HCl solution was added to the resulting filtrate to adjust its PH to 4.5. The same procedure as that of above was repeated to obtain a filtrate and to adjust its PH to 6.5. The resulting filtrates were kept at 6 degree C. for 12 hours and then the precipitates were collected and dried to obtain 1 gm and 2 gm powders, respectively, HPLC analysis of the powders showed that hawthorn berry extracts contained rutin (0.464 gm, 0.928 gm), quercetin (0.29 gm, 0.58 gm), kaempherol (0.209 gm, 0.418 gm), vitexin (0.455 gm, 0.910 gm) and the purity was 60% and 63%, respectively.

### EXAMPLE 3

Toxicity of rutin, quercetin, kaempherol and vitexin in mice by oral administration.

24 specimens of 8 week old, with specific pathogen free, ICR female mice, each weighing from 25 to 30 gm, were divided into four groups (6 mice each) and were kept in separate cages under an environment of 23+_{—}3 degree C., relative humidity of 45+_{—}5%, and 12 light/12 dark photoperiod, fed with Harlan Teklad-2018 global rodent diet (18% protein)(Kaytee Co. Madison, Wis. USA) and water was sterilized to feed to the mice.

Rutin, quercetin, kaepherol (Aldrich-Sigma Co. St. Louis, Mo. USA) and vitexin (Indofine Co. Somerville, N.J., USA) were dissolved in 0.5% of tween-80 solution to a final concentration of 150 mg/ml, 12.5 mg/ml, 100 mg/ml and 50 mg/ml respectively, and was orally fed to the 4 separate groups of mice in an amount of 0.2 ml per 20 gm of mouse body weight, i.e., rutin 1500 mg/kg, quercetin 125 mg/kg, kaempherol 1000 mg/kg and vitexin 500 mg/kg respectively. The solution was administered once and the mice were observed for 30 days for signs of adverse effects or death according to the following schedule: 1 H, 4 H, 8 H, 12 H, (hour) after administration and then every 12 hours thereafter. The daily weight of each mouse was recorded. On day 31, the mice were sacrificed and the internal organs including liver, kidney, heart, lung, muscle, stomach, urinary bladder, intestines, pancreas and spleen were examined visually and microscopically.

All mice were alive at day 31 and no body weight loss occurred during this period of observation. The mice did not develop any pathological abnormality either visually or microscopically. Therefore, it is concluded that the hawthorn berry extract which includes rutin, quercetin, kaempherol and vitexin is not toxic when orally administered to a mammal.

### EXAMPLE 4

Administration of Hawthorn Berry Bioflavonoids; Rutin, Quercetin, Kaempherol and Vitexin to Rabbits

(Step A) 36 specimens of three month old New Zealand white rabbits (Harlan Kaytee Co. San Diego, Calif.) each weighing 2.5-to 2.6 kg were fed under a condition of temperature of 23+_{—}3 degree C., relative humidity of 45+_{—}5% and photoperiod 12 light/12 dark. The rabbits were divided into six groups with 6 heads each and were fed with six different diets; Harlan Taklad rabbit diet-TD-1376 (Madison, Wis. USA) which contained of 1% cholesterol in control group; 1% cholesterol plus 1.5 mg/kg Simvastatin (Merck Co. N.J. USA) in comparative group; 1% cholesterol plus 0.15% rutin in rutin group; 1% cholesterol plus 0.15% quercetrin in quercetin group; 1% cholesterol plus 0.15% kaempherol in kaempherol group; 1% cholesterol plus 0.15% vitexin in vitexin group.

Harlan Taklad rabbit diet TD-1376 contains moisture 12%, crude protein 16%, crude fat 2%, crude fiber 15%, ash 8%, nitrogen free substances 47%.

The rabbits were fed for 8 weeks with free access to specific high cholesterol diets and water. Body weight was recorded every 7 days and the records were analyzed. All rabbits showed a normal growth rate and there were no significant differences among the six groups in regard to the diet ingestion amount and the body weight gain. (Rutin, quercetin and kaempherol were purchased from Aldrich-Sigma Co. St. Louis, Mo. USA) (Vitexin was purchased from Indofine Co. Sumerville, N.J. USA)

(Step B) After 8 weeks of breeding, the rabbits were anesthetized with injection of ketamine 75 mg/kg in the femoral muscle and sacrificed. Blood samples were collected from the heart of each rabbit to determine the blood analysis consisting of; complete blood count (CBC), Chemistry-7 and 24, (including Liver and renal function tests), lipid profiles, (including Total cholesterol, HDL, LDL, VLDL and triglycerides), coagulation factors consisting of; Prothrombin time (PT), Partial thromboplastin time (PTT), and immuno-globulin-E (an anti-allergenic factor).

### EXAMPLE 5

Analysis of Plasma Total Cholesterol, HDL and triglycerides in Rabbits.

(Step C)The effects of administering rutin, quercetin, kaempherol, and vitexin to rabbits on plasma cholesterol and triglycerides contents were deterimined as follows.

The blood samples collected from rabbits of the six dietary groups were allowed to stand for 2 hours, then centrifuged at 4000 rpm for 10 minutes. (Megafuge, Baxter-Heraeus instrument Co. N.J., USA) The supernatants were separated and stored in a deep freeze before analysis. The chemistry analysis was carried out by blood chemistry analyzer (Cobras-Integra-700, Roche Diagnostic Lab. Indiana, USA) to determine the changes in total cholesterol, HDL, LDL, triglycerides, liver function tests (such as SGOT, SGPT, G-GPT) and coagulation factors (PT, PTT) (Bayer-MLA-Electra-900 Automatic coagulation timer). The results were tested by using student t-test and Microsoft excel-7.0 program. The results are depicted in Table III.

3 TABLE III.

Blood analysis on rabbits fed on 6 different high cholesterol diets

Control group, Simvastatin group, Rutin group, Quercetin group, kaempherol group, vitexin group.

Control group; TC; (383.3+_{—}55.2 mg/dl), TRG (165.6+_{—}40.6 mg/dl), HDL (45.6+_{—}22.4 mg/dl), SGOT (35+_{—}6 u/l), SGPT (62.5+_{—}6.5 u/l), GGTP (5+_{—}2 u/l); WBC; (4.8+_{—}2.0 k/ul), A; (33+_{—}10), B; (3.5+_{—}0.5).

Simvastatin group; TC;(277.3+_{—}90.7), TRG; (141+_{—}30), HDL; (47.5+_{—}16.5), SGOT; (114.3+_{—}30.7), SGPT; (71.2+_{—}3.8), GGTP; (6+_{—}1); WBC; (3.3+_{—}2.6 k/ul), A; (13.5+_{—}6.5), B; (2.8+_{—}0.3).

Rutin group; TC; (254.5+_{—}36.5), TRG; (108+_{—}22), HDL; (36+_{—}6), SGOT; (52.8+_{—}12.2), SGPT; (33+_{—}9), GGTP; (3+_{—}1), WBC; (4.8+_{—}1.2), A; (11.3+_{—}6), B; (2.6+_{—}0.4).

Quercetin group: TC; (262.3+_{—}30.7), TRG; (105.6+_{—}18.4), HDL; (42+_{—}8), SGOT; (62+_{—}7), SGPT; (43+_{—}12),GGTP; (4+_{—}1), WBC; (4.5+_{—}1.5). A;(12.8+_{—}5.4), B; (2.7+_{—}0.3).

Kaempherol group; TC; (275+_{—}23), TRG; (130+_{—}26), HDL; (45.3+_{—}7.3), SGOT(65+_{—}8.6), SGPT; (42+_{—}8.8), GGTP; (3.2+_{—}1.8), WBC; (4.6+_{—}1.8), A; (13.5+_{—}4.6), B; (2.8+_{—}0.4).

Vitexin group; TC; (269.5+_{—}38.5), TRG; (138+_{—}28), HDL; (46+_{—}12), SGOT; (60+_{—}12.5), SGPT; (48+_{—}15), GGTP; (3+_{—}1.5), WBC; (4.9+_{—}1.7), A; (13.7+_{—}4.3), B; (2.9+_{—}0.5).

TC: Total Cholesterol, TRG: Triglycerides, WBC: White Blood Cell,

HDL: high density lipoprotein, A: percentage (%) proportion of fatty streak to total aortic area, B: percentage (%) proportion of fat containing cells.

From the data of Table III, administration of rutin and quercetin, kaempherol and vitexin decreased plasma total cholesterol and triglycerides by 32-33% and 45-47%, also 30-30% and 22-17%, respectively, as compared to that of control group. Rutin, quercetin, kaempherol and vitexin are more effective in reducing plasma total cholesterol and triglycerides than Simvastatin, furthermore, liver function and WBC are not affected as that of the Simvastatin group.

(Step D) Analysis for fatty streak deposition in thoracic aorta.

The chest wall of each rabbit (sacrificed in Step B) was incised, a portion of thoracic aorta from 1 cm site above aortic valve downward for 5 cm was cut out. The surrounding fatty tissues were removed, the aorta was incised longitudinally and pinned to a dish. The moist aorta was then photographed, and staining of fatty streak was carried out with the method described by Esper, E. et al. (Journal of Lab. Clinical Medicine; 121, pp 103-110(1993)) as follows.

The opened portion of aortas were pinned to a wooden tongue depressor, washed three times for 2 minutes with anhydrous propylene glycol and stained for 30 minutes with saturated solution of Oil Red O dissolved in propylene glycol. Then the aortas were washed twice for 3 minutes with 85% propylene glycol, to remove remaining staining solution by washing with normal saline solution. The aortas were photographed, and the photographs were traced with an image analyzer (LEICA, Q-600, Germany), and the area of stained portion were fatty streak region, its proportion in percentile (%) to the total aorta area were calculated. The results were shown in Table III.

FIGS. IA, IB, IC, ID, IE, IF show that the aortas of the rabbits administered with 1% cholesterol control group; 1% cholesterol plus 1.5 mg/kg simvastatin in comparative group; 1% cholesterol plus 0.15% rutin group; 1% cholesterol plus 0.15% quercetin group; 1% cholesterol plus 0.15% kaempherol group; 1% cholesterol plus 0.15% vitexin group respectively. The microscopic pictures which showed a thick layer of macrophage lipid cell complex in the arota walls of IA, but no or a very thin layer of macrophage lipid cell complex in the aorta walls of IB, IC, ID, IE, IF.

It is concluded that the rutin, quercetin, caempherol and vitexin in the present invention can prevent and/or inhibit the deposition of macrophage-lipid cell complex onto arterial endothelial walls. Therefore, long term administration would prevent and inhibit atherosclerosis and arteriosclerosis induced cardiovascular diseases and prevent formation of fatty liver as depicted on Table III. The results were tested by using student t-test and Microsoft excel-7.0 program.

(Step E) Pathological examination of rabbit organs.

Internal organs from the rabbits sacrificed in Step B including aorta, lung, heart, liver, kidney, muscle, bladder and pancreas were visually examined and showed no abnormalities. One half of each organ was frozen and the other half was fixed with 10% neutral buffered formalin solution for 24 hours. Then the fixed organs were washed with tap water and stepwise dehydrated with 70%, 80%, 90%, 100% ethanol, then embedded in paraffin by using Shandon, Histocentre-2 The embedded organ blocks were sectioned in 4 mu.m thickness with a microtome (McBain, M 820, American Optical Co. USA) and stained with hematoxylin and eosin stain (H. E. stain). Then the stained specimens were made transparent with xylene, and mounted with permount on microslides. There were no pathological abnormalities or lesions under microscopic examination.

### EXAMPLE 6

Inhibition and Prevention of Fatty Degeneration in the Liver of the Rabbits.

According to the report of Fogt F. and Nanji A., (Toxicology and Applied Pharmacology; 136, pp 87-93, 1996) and Keegan A. et al., (Journal of Hepatology; 23, pp 591-600, 1995). Fatty degeneration of liver can be classified into four grades based upon the abnormal fat containing cells around the central vein of liver acinus, for example: grade 1 (0-25%), grade 2 (26-50%), grade 3 (51-75%), grade 4 (76-100%). The effects of rutin, quercetin, kaempherol and vitexin on liver tissue on the rabbits from Example 4 Step B were examined. The results are depicted in Table III.

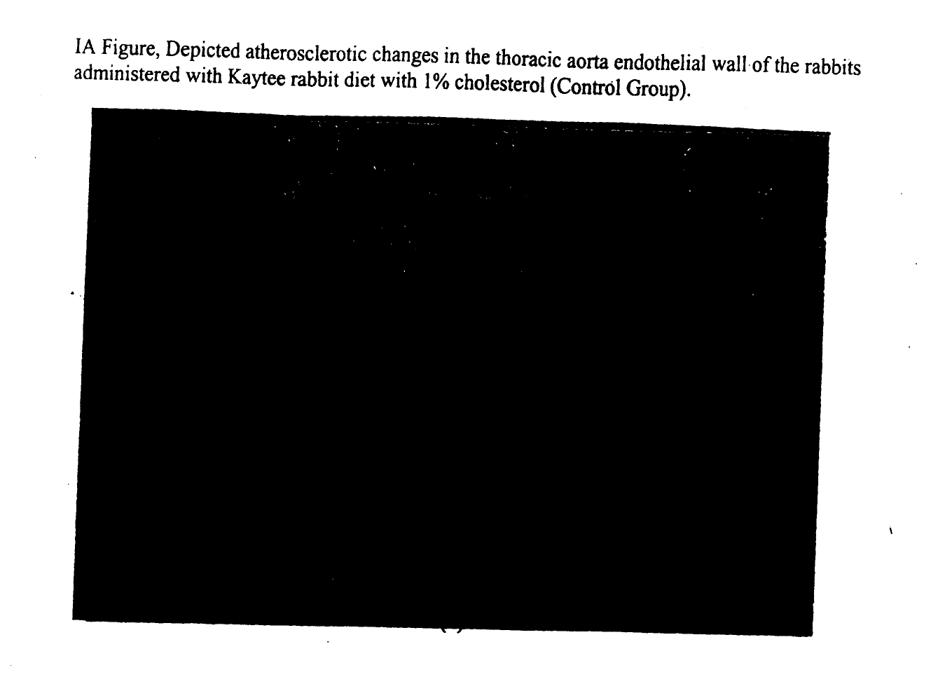

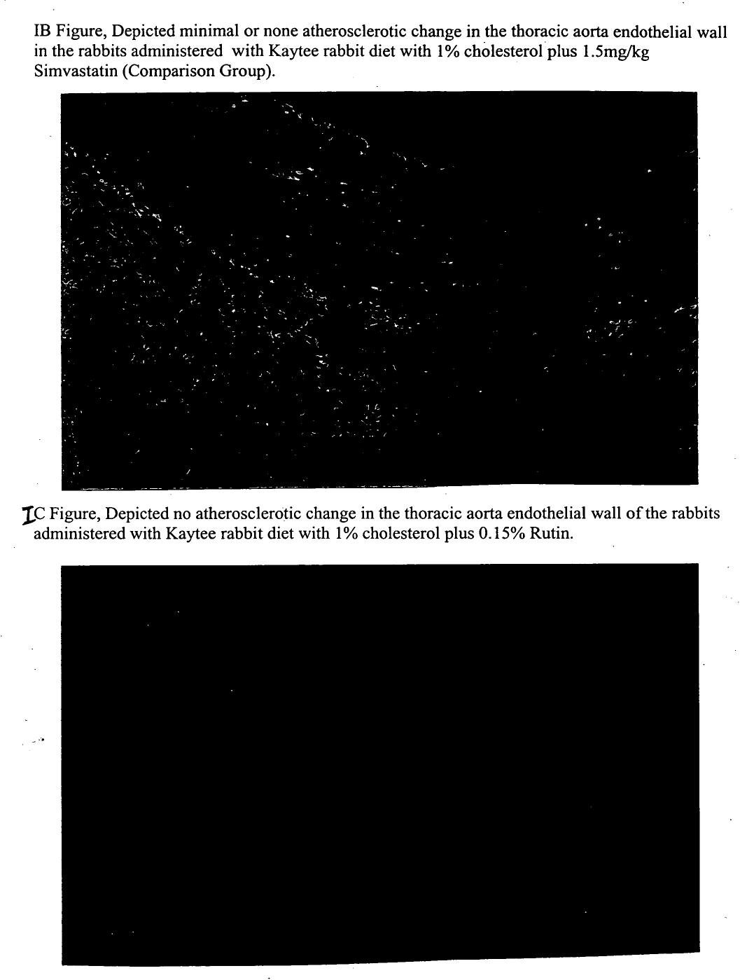

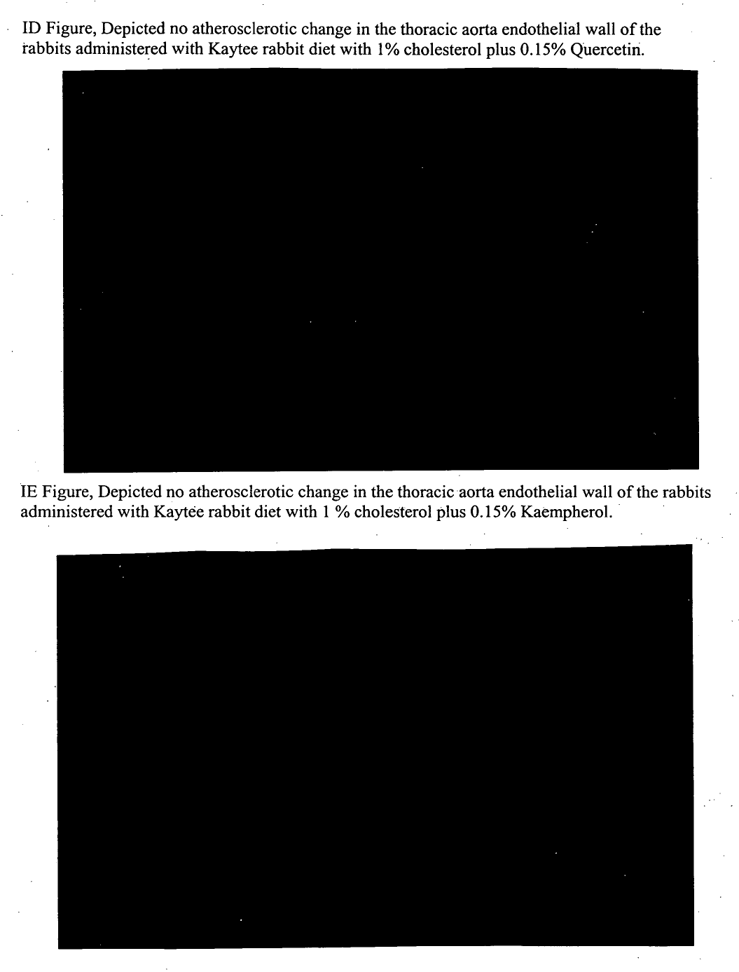

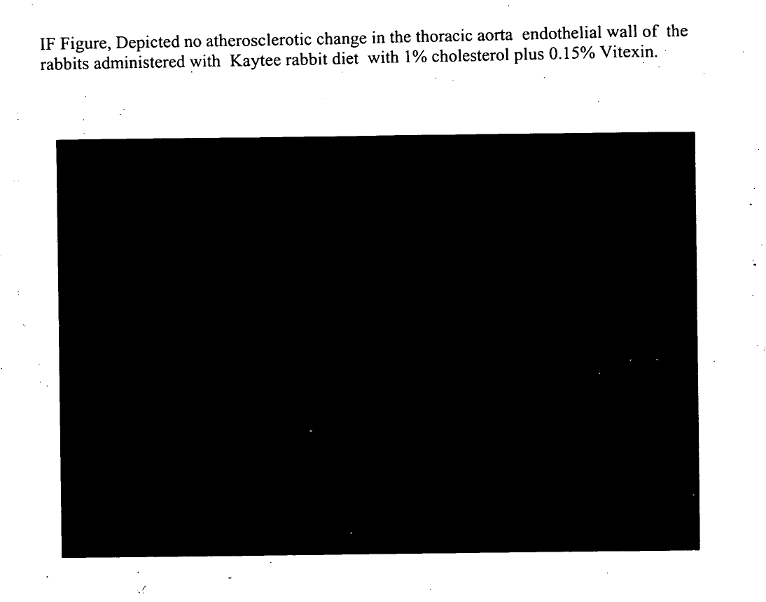

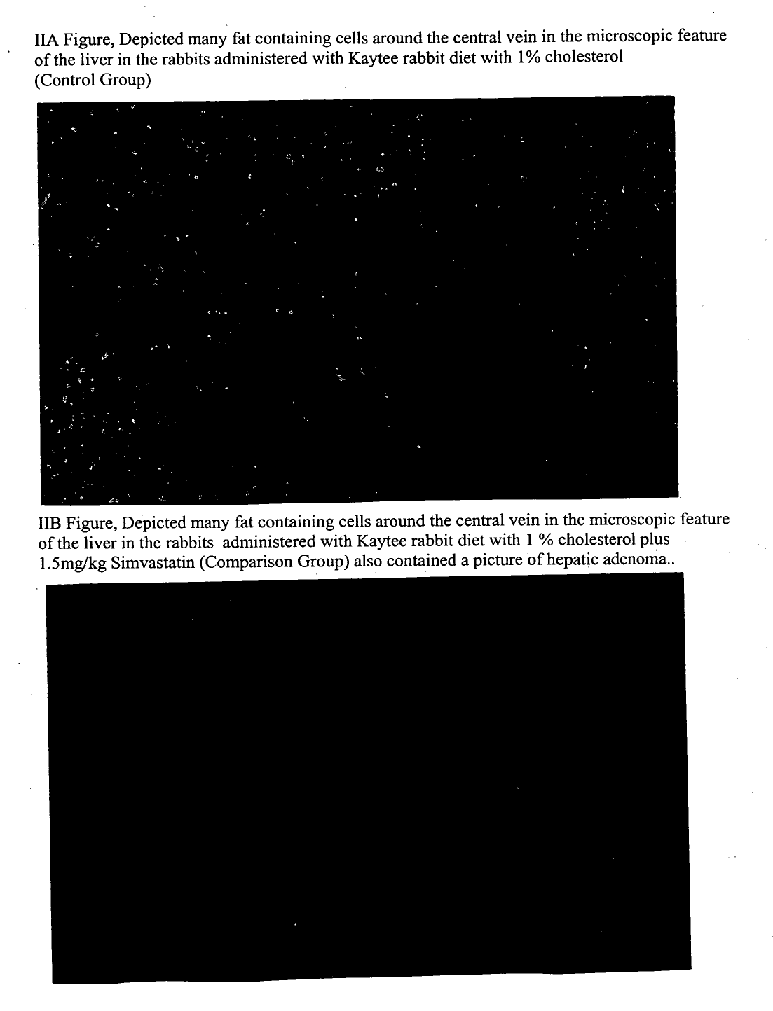

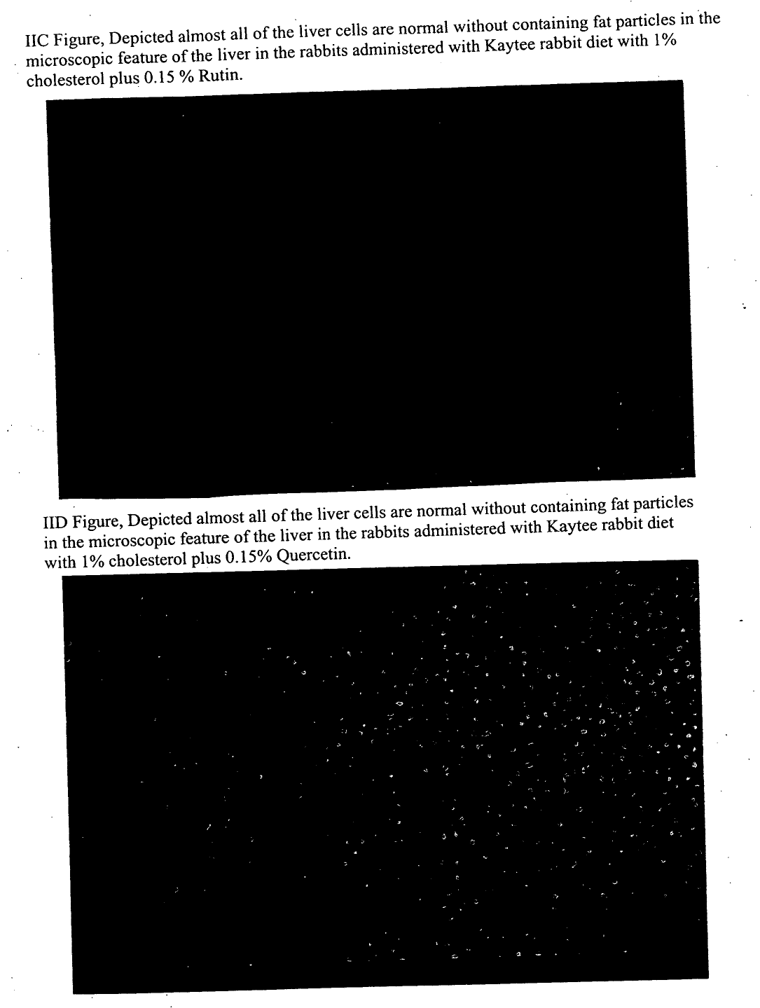

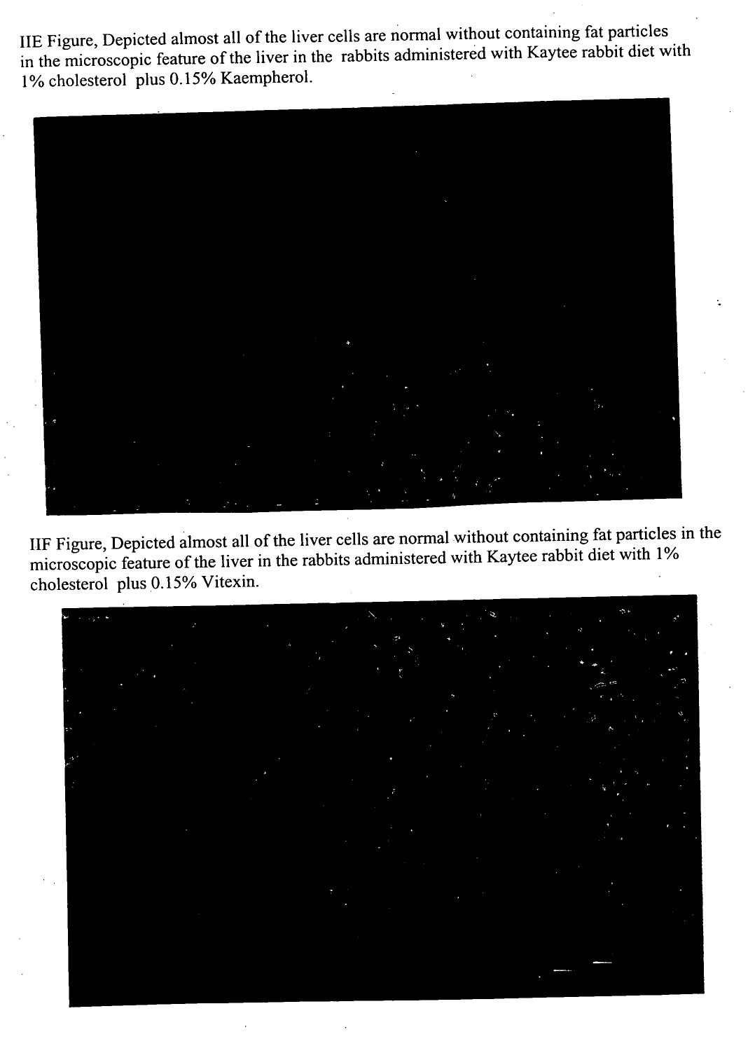

FIGS. 2A, 2B, 2C, 2D, 2E and 2F represent the microscopic features of the liver of the rabbits administered with 1% cholesterol; 1% cholesterol plus 1.5 mg/kg Simvastatin; 1% cholesterol plus 0.15% rutin; 1% cholesterol plus 0.15% quercetin; 1% cholesterol plus 0.15% kaempherol and 1% cholesterol plus 0.15% vitexin respectively. The features of 2A and 2B show many fat containing cells around the central vein. The other features in 2C, 2D, 2E and 2F showed that almost all of the liver cells are normal without containing fat particles. It depicts and shows that rutin, quercetin, kaempherol and vitexin can inhibit and prevent formation of fatty liver and fatty degeneration. Further more, one of 2B contains hepatic adenoma picture. This depicted that Simvastatin has serious side effect to the liver even with short term administration.

### EXAMPLE 7

HMG-CoA Reductase Inhibited by Rutin, Quercetin, Kaempherol and Vitexin.

The activity of 3-HMG-CoA reductase was determined by modified Hulcher's method (Journal of lipid research; 14, 625-641, 1973), in which the concentration of Co-A will be produced when 3-HMG-CoA is reduced to mevalonate salt by the action of the 3-HMG-CoA reductase; then the reductase was determined by spectroscopy and the activity of the 3-HMG-CoA reductase was calculated.

Preparation of Microsomes from the Liver Tissues.

Liver tissues from each group of rabbits from Example 4 Step B were obtained, 3 gram of tissue was washed with 100 ml of cold normal saline (1 N NaCl) and 100 ml of cold buffer solution-A (0.1M triethanolamine HCl/10 mM EDTA/10 mM dithiothreitol (DTT)). A cold buffered solution-A and liver tissue ratio was 2 ml per each gram. Then the mixture was minced and homogenized with a homogenizer, the homogenate was centrifuged at 20,000 times gram for 15 minutes, the supernatant was ultracentrifuged at 100,000 times gram for 60 minutes and again for another 60 minutes to obtain microsomal precipitates. The precipitates were washed with cold buffer solution-A and kept at minus 70 degree C. till ready for analysis.

Assay of the Activity of 3-HMG-CoA Reductase.

There were 3 reactive substrates used in this assay (a) buffer solution B; 0.1 M triethanolamine HCl/10 mM EDTA with ph 7.4 (b) 3-HMG-CoA solution; 150 mu.moles/culture medium, (c) NADPH solution; 2 mu.moles/culture medium.

The microsomal suspension was then mixed with reaction substrates in a centrifuge tube and allowed to react at 37 degree C. for 30 minutes, then added with 20 mu 1 of 10 mM sodium arsenous solution and allowed to react for 1 minute, and then added with 100 mu.l of citrate buffer solution (2M sodium citrate/3 M sodium tungstate, with Ph of 3.5) at 37 degree C. for 10 minutes, then centrifuged at 30,000 times gram for 15 minutes to remove protein. Then 1 ml of this supernatant was transferred to a tube with a cap and added with 0.1 ml of 2M tris-hydrochloride solution (Ph 10.6) and 0.1 ml of 2M tris-hydrochloride solution (Ph 8) to adjust its ph to 8.0.

The mixture was then added with 20 mu.l of DTNB buffer solution (3 mM DTNB/0.1M triethanolamine/10 mM EDTA with PH of 7.4). The absorbance of this mixture was determined at wave length of 412 nm to calculate the amount of CoA that is the activity of 3-HMG-CoA reductase. The amount of inhibition on the activity of the 3-HMG-CoA reductase by rutin, quercetin, kaempherol and vitexin were calculated base on the above method. The results are depicted in Table IV.

4 TABLE IV; Inhibition on the activity of 3-HMG-CoA reductase in percentage (%); control group: 0%, rutin group: 45%, quercetin group: 32%, Kaempherol group: 30%, vitexin group: 30%.

From the above depiction, the Rutin, Quercetin, Kaempherol and Vitexin all exhibit inhibition on the activity of the 3-HMG-CoA reductase between 45-30%.

### EXAMPLE 8

Extraction of Rutin, Quercetin, Kaempherol and Vitexin from Hawthorn Berry.

Dried hawthorn berry in an amount of 300 gm was added to 500 ml of 0.5 N of Ca (OH) sub.2 solution and adjusted its pH to 10-12 at room temperature, then stood for 12 hours, and the pH was adjusted to 6-7. The precipitate was recovered then dried to obtain a powder mixture of crude rutin, quercetin, kaempherol and vitexin with purity about 40%.

Another method was by using 500 ml of 80% ethanol added to 300 gram of dried hawthorn berry at a temperature of 60 degree C. for 6 hours, and then the mixture was filtrated and resulting extract contained about 5% rutin, 3% quercetin, 2% Kaempherol and 0.5% vitexin.

### EXAMPLE 9

Effects of Rutin, Quercetin, Kaempherol, and Vitexin in Human by Oral Administration.

Sixty women ranging from their fourth to sixth decades of age were randomly divided into three groups (20 persons in each group); (a) one group was on hawthorn berry extract powder of a daily dose of 5 gram dissolved in water for 30 days, (b) one group was on rutin 50 mg/kg/day, quercetin 10 mg/kg/day and Vitamin C 5 mg/kg in the form of capsule for 30 days, (c) another group was on kaempherol 50 mg/kg/day and vitexin 1 mg/kg/day and Vitamin C 10 mg/kg for 30 days. The plasma total cholesterol, HDL,LDL, Triglycerides, CBC, WBC, SGOT, SGPT, GGTP, BUN (blood urea nitrogen), Creatinine (Cr), PT, PTT, and Immuno-globulin-E contents were determined before and after administration of above substances. The results are depicted in TableV. (the results were tested by using student-t test and Microsoft-excel 7.0 program) 5 TABLE V. Blood analysis in 60 human volunteers on rutin, quercetin, kaempherol and vitexin administration.

Hawthorn berry extract group; (80% effectively decreased TC and TrG), TC (−20 to −30%), TrG (20 to −25%), HDL (−15 to +15%), LDL (−25% to −30%), SGOT (−25 to −35%), SGPT (−20 to −35%), GGTP (−10 to +10%), WBC (−15 to −20%), BUN (−10%), Cr (−5 to −10%), PT (+8 to +20%), PTT (+15 to +20%), IGE (−15 to +15%.)

Rutin and quercetin group; (85% effectively decreased TC and TrG), TC (−20 to −30%), TrG (−20 to −30%), HDL (+20 to+25%), LDL (−30% to−32%), SGOT (−25 to −35%), SGPT (−20 to −35% GGTP (−10 to +10%), WBC (−10 to −20%), BUN (−10 to −20%), Cr. (−5 to −10%), PT (+10 to+20%), PTT (+15 to+20%), IGE (−15 to+15%).

Kaempherol and vitexin group; (80% effectively decreased in TC and TrG), TC (−20 to −30%), TrG (−20 to −25%), HDL (+20 to +25%), LDL (−25% to 26%), SGOT (−25 to −35%), SGPT (−20 to −35%), GGTP (−10 to +10%), WBC (−10 to −20%), BUN (−10 to −20%), Cr.(−5 to −10%), PT (+10 to +20%), PTT (+15 to +20%), IGE (−15 to +15%).

TC; total cholesterol, TrG; triglycerides,

From Table V, it shows that administration of hawthorn berry extract, rutin-quercetin and kaempherol-vitexin plus Vitamin C in humans not only result in decreased total plasma cholesterol and triglycerides in 80 to 85% of subjects studied (amount of decreased were 25% and 20% respectively,)and increased in serum high density lipoprotein (HDL) by at least 20%. but also improved liver and kidney functions. There was only mild leukopenic effect, mild inhibition on coagulation and inhibition of allergic reaction.Therefore, administration of Rutin, Quercetin, Kaempherol, Vitexin and /or a mixture thereof will sufficiently prevent and/or inhibit the formation of hyperlipidemia, atherosclerosis and ateriosclerosis. They also prevent incident of stroke and embolization by decreasing coagulation of blood due to prolonging of PT and PTT.

### EXAMPLE 10

Effects on Plasma Lipid Metabolism in Rabbits by Oral Administration of Hawthorn Berry Extract.

15 specimens of two month old New Zealand white rabbits, each weighing from 2.5 to 2.6 kg, were fed under a temperature of 23+_{—}3 degree C., relative humidity of 45+_{—}5 % and photoperiod of 12 light/12 dark, and were fed with Kaytee global rabbit diet (Kaytee Co., Chilton, Wis. USA) (containing moisture 12%, crude protein 16%, crude fat 2%, crude fiber 18%, nitrogen free substances 47.7%, minerals and vitamins 1.25%). Animals were allowed to free access to food and water and were fed for 4 weeks.

The hawthorn berry extract obtained from Example-2 (A) was dissolved in 0.5% Tween 80 solution to a concentration of 100 mg/ml, and was given orally to the rabbits in an amount of 50 mg/kg/day, one group was given hawthorn berry extract in an amount of 50 mg/kg/day, plus Vitamin C 10 mg/kg/day for 4 weeks. Blood samples from the external jugular vein were obtained for lipid analysis before and after administration of hawthorn berry extract. The results of the changes are depicted in Table VI.

6 TABLE VI. Effects on plasma lipids in rabbits by hawthorn berry extract Before administration; TC (55.2+_{—}6.3), TrG. (95.6+_{—}23.4), HDL (29.5+_{—}6.5). After administration; TC (43.2+_{—}4.2), TrG. (68.6+_{—}12.4), HDL (25.6+_{—}6.4). Hawthorn berry extract and Vitamin C; TC (43.1+_{—}3.8), TrG (66.8+_{—}11.4), HDL (36+_{—}5.6).

Again, this is evidence that administration of hawthorn berry extract in rabbits reduced plasma total cholesterol and triglycerides by 22% and 28% respectively and increased HDL by 20%. Liver and kidney function, white blood cells were not affected. (data are not depicted).

### EXAMPLE 11

Pharmaceutical Formulation and Preparation

Hard and/or soft gelatin capusles are prepared and the ingredients are as follows:

Formulation 1

Quantity (mg/capsule): Active ingredient 200 (Rutin, quercetin, kaempherol, and vitexin or a mixture), Vitamin C 200, Starch or Lactose (carrier) 100, Total 500 mg.

Formulation 2

Quantity (mg/capsule): Active ingredient 100 (hawthorn berry extract of Example 2-A), Vitamin C 100, Starch or Lactose (carrier) 300, total 500 mg.

### EXAMPLE 12

Foods Containing Hawthorn Berry Powder or Extract or Rutin, Quercetin, Kaempherol, and Vitexin or a Mixture Thereof, Plus Vitamin C.

Foods containing above active ingredients are prepared as follows:

(1) Preparation of Tomato Ketchup and Sauces

A hawthorn berry powder with particle size ranging from 100 to 200 mu.m was added to a tomato ketchup or sauce in a sufficient amount to achieve a health improving tomato ketchup or sauce containing 0.01 to 10 wt % of rutin, quercetin, kaempherol, vitexin or a mixture thereof, plus 0.1 to 1 wt % of Vitamin C.

Alternatively, the hawthorn berry extract obtained in Example 8 was added to a tomato ketchup or sauce to achieve a health improving tomato ketchup or sauce containing 0.01 to 10 wt % of rutin, quercetin, kaempherol, vitexin or a mixture thereof plus 0.1 to 1 wt % of Vitamin C.

(2) Preparation of Foods Containing Wheat and Cereal Flour

A hawthorn berry powder was added to wheat and cereal flour in a sufficient amount to achieve a wheat flour mixture containing 0.01 to 10 wt % of rutin, quercetin, kaempherol, vitexin or a mixture thereof plus 0.1 to 1 wt % of Vitamin C. These mixtures of the wheat or cereal flour are prepared for breads, cakes, cookies, crackers and noodles as health improving foods.

Alternatively, these foods were prepared by using a wheat flour mixture containing 0.01 to 10 wt % of rutin, quercetin, kaempherol, vitexin or a mixture in form of hawthorn berry extract obtained in Example 8.

(3) Preparation of Soups and Gravies.

A hawthorn berry powder was added to soups and gravies in a sufficient amount to achieve health improving soups and gravies containing to 10 wt % of rutin, quercetin, kaempherol, vitexin or a mixture thereof, Plus 0.1 to 1 wt % of Vitamin C.

Alternatively, these foods were prepared by using the hawthorn berry extract in Example 8.

(4) Preparation of Ground Beef.

A hawthorn berry powder was added to ground beef in a sufficient amount to achieve health improving ground beef containing 0.01 to 10 wt % of rutin, quercetin, kaempherol, vitexin or a mixture thereof, plus 0.1 to 1 wt % of Vitamin C.

Alternatively, these foods were prepared by using the hawthorn berry extract in Example 8.

(5) Preparation of Dairy Products.

A hawthorn berry powder was added to milk in a sufficient amount to achieve the milk containing 0.01 to 10 wt % of rutin, quercetin, kaempherol, vitexin or a mixture thereof, Plus 0.1 to 1 wt % of Vitamin C. Other various dairy products such as butter and ice cream were prepared therefrom.

Alternatively, these foods were prepared by using the hawthorn berry extract in Example 8.

In case of cheese and yogurt preparation, a hawthorn berry powder or extract was added to coagulated milk protein after fermentation.

(6) Preparation of Beverages

A hawthorn berry powder or extract was added to Coca-Cola, Pepsi-Cola or other carbonated drink or beer and alcoholic beverages in a sufficient amount to achieve the beverage containing 0.01 to 10 wt % of rutin, quercetin, kaempherol, vitexin or a mixture thereof. Plus 0.1 to 1 wt % of Vitamin C.

(7) Preparation of Fruit Juice

10 to 100 grams of the hawthorn berry powder or extract obtained in Example 8 and 500 to 1000 mg of Vitamin C were added to each 1000 ml of a fruit juice to achieve a health improving fruit juice.

(8) Preparation of Vegetable Juice

10 to 100 grams of the hawthorn berry powder or extract obtained in Example 8 and 500 to 1000 mg of Vitamin C were added to each 1000 ml of a vegetable juice to achieve a health improving vegetable juice

(9) Preparation of the Health Foods that Containing Rutin, Quercetin, Kaempherol, Vitexin or a Mixture Thereof.

An improved health food was prepared by mixing the following ingredients and tabletting or capsuletting the mixture.

Quantity (wt/wt %): Active ingredients; (rutin, quercetin, kaempherol, vitexin or a mixture thereof) 20%, Vitamin C 20%, Ginseng root powder or extract 40%, Carrier and flavor 20% equals total of 100%.

An improved health food was prepared by mixing the following ingredients and tabletting or capsuletting the mixture.

Formulation 3

Quantity(mg/tablet, or mg/capsule): Active ingredients; (rutin, quercetin, kaempherol, vitexin or a mixture thereof) 200 mg, and a typical daily multivitamin tablet: calcium carbonate, ascorbic acid (200 mg, 333% RDI), gelatin, vitamin E acetate (30I.U., 100% RDI), starch, niacin amide (20 mg, 100% RDI), hydroxypropyl-methyl-cellulose, calcium pantothenate (10 mg,100% RDI), selenium yeast, polyvinyl-pyrolidone, hydroxyl-propyl-cellulose, manganese sulfate, calcium silica, pyridoxine hydrochloride (2 mg, 100% RDI), riboflavin (1.7 mg, 100% RDI), thiamine mononitrate (1.5 mg, 100% RDI), beta carotene & vitamin A acetate (5000 I.U., 100% RDI), sodium hexameta-phosphate, biotin (30 mu.g, 10% RDI), vitamin D (400 I.U., 100% RDI), vitamin B-12 (cyanocobalamin, 6 mu.g, 100%), and lecithin.

Formulation 4

Quantity (mg/tablet, or mg/capsule): Active ingredients; (rutin, quercetin, kaempherol, vitexin or a mixture thereof) 200 mg, and a typical daily multivitamin and minerals tablet which contains same vitamins and amount as listed above in Formulation 3. Calcium phosphate (130 mg of elemental calcium), magnesium hydroxide & stearate (100 mg, 25% RDI), cellulose, potassium chloride, gelatin, ascorbic acid (200 mg, 333% RDI), ferrous fumarate (18 mg of elemental iron, 100% RDI), zinc sulfate (15 mg, 100% RDI), modified cellulose gum, vitamin E acetate (30 I.U., 100 % RDI), citric acid, starch, niacin amide (20 mg, 100% RDI), hydroxyl-propyl-methyl cellulose,calcium pantothenate (10 mg, 100% RDI), selenium yeast, polyvinylpyrolidone, hydroxyl-propyl-cellulose, manganese sulfate, silica, copper oxide (2 mg, 100% RDI), chromium yeast, molybdenum yeast, pyridoxine hydrochloride (vitamin B-6, 2 mg, 100% RDI), riboflavin (vitamin B-2, 1.7 mg, 100% RDI), thiamine mononitrate (vitamin B-1, 1.5 mg, 100% RDI), beta carotene & vitamin A acetate (5000 I.U., 100% RDI), potassium iodide (150 mu.g, 100% RDI), sodium hexameta-phosphate, biotin (300 mu.g, 100% RDI), vitamin D (400 I.U., 100% RDI), lecithin, and vitamin B-12 (cyanocobalamin, 6 mu.g, 100% RDI).

Formulation 5

Quantity (mg/tablet or mg/capsule): Active ingredients; (rutin, quercetin, kaempherol, vitexin or a mixture thereof) 200 mg, and a typical daily multivitamin and minerals tablet for older adults which contains same vitamins and amount of minerals as listed in Formulation 4, but contains larger amount in following substances; calcium carbonate, calcium phosphate (200 mg Ca, 20 % RDI, 48 mg phosphorous, 5% RDI), magnesium stearate & oxide (100 mg, 25% RDI), potassium chloride (80 mg, 2 % RDI), microcrystalline cellulose, ascorbic acid (200 mg, 333% RDI), gelatin, d-1-alpha-tocopheryl acetate (45 I.U., 150% RDI), modified food starch, maltodextrin, crospovidone, reduced iron (4 mg, 22 % DI),hydroxyl-propyl-methylcellulose, niacin amide (20 mg, 100% RDI), zinc oxide (15 mg, 100% RDI), calcium pantothenate, manganese sulfate(3.5 mg), vitamin D (400 I.U., 100% RDI), titanium dioxide, vitamin A and beta.-carotene (5000 I.U., 100% RDI), stearic acid, Pyridoxine HCl (3 mg, 150% RDI), nickelous sulfate (5 mu.g), phytonadione, and chromium chloride (130 mu.g), ascorbic acid (200 mg, 333% RDI), silicon dioxide, copper oxide (2 mg, 100% RDI), dextrose, thiamine mononitrate (1.5 mg, 100% RDI), triethyl citrate, polysorbate 80,chromium chloride (130 mu.g), artificial color, potassium iodide (150 mu. g, 100% RDI), sodium metasilicate (2 mg), sodium molybdate (160 mu.g), borates, sodium selenate (20 mu.g), biotin (30 mu.g, 10% RDI), sodium metavanadate (10 mu.g), lecithin and cyanocobalamin (vitamin B-12, 25 mu.g, 417% RDI).

The total daily dose of the compounds of this invention administered to a human in a single or in divided doses can be in an amount, for example, of 0.1 to 500 mg/kg body weight daily or more preferably from about 1 to 100 mg/kg body weight for oral administration or 0.1 to about 10 mg/kg for parenteral administration daily.

The amount of active ingredients that may be combined with the carrier materials to produce a single dosage form will vary depending upon the host treated and the particular mode of administration.

While the present invention has been described with respect to the above specific descriptions and embodiments, it should be recognized that various modifications and changes may be made to the present invention by those skills in the art which also fall within the scope of the invention as defined by the appended claims and their legal equivalents.

What is claimed is:

1. The present invention relates to a method for inhibiting cholesterol synthesis through inhibiting the activity of 3-hydroxy-3-methyl-glutaryl-Co-enzyme A (3-HMG-CoA) reductase in a mammal, which comprises administering thereto an effective amount of bioflavonoids derived from fructus crataegus (Crataegus Oxyacantha) such as rutin, quercetin, kaempferol, vitexin or a mixture thereof.

2. The method of claim 1: wherein the mammal is human.

3. The method of claim 1: wherein the rutin, quercetin, kaempferol, vitexin or a mixture thereof is administered in the form of pharmaceutical composition containing an effective amount of rutin, quercetin, kaempferol, vitexin or mixture thereof and pharmaceutically acceptable excipients, carriers or diluents.

4. The method of claim 1, wherein the effective amount of the rutin, quercetin, kaempferol, vitexin or a mixture thereof ranges from 0.1 to 500 mg/kg of body weight/day.

5. The method of claim 1, wherein the rutin, quercetin, kaempferol, vitexin or a mixture thereof is administered in a form of a health improving food composition containing rutin, quercetin, kaempherol, vitexin or a mixture thereof in an amount ranging from 0.01 to 10 wt %

6. The method of claim 5, wherein the rutin, quercetin, kaempferol, vitexin or a mixture is added in the form of a powder or an extract from hawthorn (Crataegus Oxyacantha) fruit, flower, leaf, stem, or various mixtures of different plant parts.

7. The method of claim 5, wherein the food is meat, confectionery (such as chocolate and gum), pizza, a food product made from cereal flour, dairy products, soups, broth, pastes, sauces (such as ketchup), vitamin complexes, or health food.

8. The method of claim 7, wherein the food product made from cereal flour is bread, cake, cracker, cookie, biscuit or noodle.

9. The method of claim 7, wherein the dairy product is milk, ice cream, slush, cheese or yogurt.

10. The method of claim 1, wherein the rutin, quercetin, kaempferol, vitexin or a mixture thereof is administered in the form of a health improving beverage composition containing rutin, quercetin, kaempferol, vitexin or a mixture thereof in an amount ranging from 0.01 to 10 wt %.

11. The method of claim 10, wherein the beverage is a fruit juice, vegetable juice, tea, carbonated beverage or alcoholic beverage.

12. The method of claim 1, the bioflavonoids derived from hawthorn berry (Fructus Crataegus) extract is prepared by extracting dried hawthorn berry with a solvent selected from the group consisting of water, a low alcohol and an aqueous alkali- or alkaline earth-metal hydroxide solution.

13. The method of claim 1, wherein the said composition further includes vitamin C.

14. The method of claim 13, wherein the vitamin C is administered at a dose ranging from 5-20 mg/kg of body weight per day.

15. The method of claim 3, wherein the serum total cholesterol and triglycerides were decreased by greater than 25% and 35% respectively.

16. The method of claim 14, wherein the high density lipoprotein cholesterol (HDL) is increased by greater than 20%.
